(19) United States
(12) Patent Application Publication
(10) Pub. No.: US 2024/0082296 A1
(43) Pub. Date: Mar. 14, 2024

(54) TREATMENT OF MUSCLE ATROPHY USING DEXTRAN SULFATE

(71) Applicant: TX MEDIC AB, Viken (SE)

(72) Inventor: Lars BRUCE, Viken (SE)

(21) Appl. No.: 18/246,569

(22) PCT Filed: Oct. 7, 2021
(86) PCT No.: PCT/SE2021/050983

§ 371 Date: Mar. 24, 2023

(30) Foreign Application Priority Data
Oct. 9, 2020 (SE) 2051180-4

(51) Int. Cl. A61K 31/737; A61P 21/00; A61P 25/28

(52) U.S. Cl. CPC A61K 31/737; A61P 21/00; A61P 25/28

(57) ABSTRACT

The present invention relates to the use of dextran sulfate, or a pharmaceutically acceptable salt thereof, in treatment or prevention of muscle atrophy in a subject suffering from sarcopenia and in improving muscle function in a subject suffering from a neuromuscular disease and/or damage or sarcopenia.

### TECHNICAL FIELD

The present invention generally relates to muscle atrophy, and treatment or prevention thereof using dextran sulfate, or a pharmaceutically acceptable salt thereof.

### BACKGROUND

Muscle atrophy is the loss of skeletal muscle mass. Muscle atrophy can be caused by immobility, aging, malnutrition, medications, or a wide range of injuries or diseases that impact the musculoskeletal or nervous system. Muscle atrophy leads to muscle weakness and causes disability.

Disuse causes rapid muscle atrophy and often occurs during injury or illness that requires immobilization of a limb or bed rest. Depending on the duration of disuse and the health of the individual, this may be fully reversed with activity. Malnutrition first causes fat loss but may progress to muscle atrophy in prolonged starvation and can be reversed with nutritional therapy. Sarcopenia is the muscle atrophy associated with aging and is thought to be primarily a disease of mitochondrial dysfunction and can be slowed by exercise. Finally, genetic diseases of the muscles, such as muscular dystrophy or myopathies, can cause atrophy, as can damage to the nervous system (neuropathies), such as in spinal cord injury or stroke.

Whatever the initiating cause, muscle atrophy results from an imbalance between protein synthesis and protein degradation, although the precise mechanisms are incompletely understood. Current treatment depends on the underlying cause but will often include exercise and adequate nutrition in particular for muscle atrophy caused by immobility, aging or malnutrition. Anabolic agents may have some efficacy but are not often used due to side effects. There are multiple treatments and supplements under investigation but there are currently limited treatment options in clinical practice.

Hence, there is a need for a treatment or prevention of muscle atrophy that improves muscle function in a subject.

### SUMMARY

It is a general objective to treat or prevent muscle atrophy.

It is another general objective to improve muscle function in subjects suffering from a neuromuscular disease or damage.

These and other objectives are met by embodiments as disclosed herein.

The present invention is defined in the independent claims. Further embodiments of the invention are defined by the dependent claims.

An aspect of the invention relates to dextran sulfate, or a pharmaceutically acceptable salt thereof, for use in treatment or prevention of muscle atrophy in a subject suffering from sarcopenia.

Another aspect of the invention relates to dextran sulfate, or the pharmaceutically acceptable salt thereof, for use in improving muscle function in a subject suffering from a neuromuscular disease and/or damage or sarcopenia.

Dextran sulfate treatment arrested muscle atrophy and improved muscle function in patients suffering from amyotrophic lateral sclerosis (ALS). The results of the dextran sulfate treatment included enhanced physical activity of the ALS patients. Furthermore, dextran sulfate did not only reduce muscle degeneration as seen in a significant reduction in serum creatine kinase and myoglobin and a significant increase in serum concentration of alanine but also improved muscle function and activity as seen in significantly raised levels of serum lactate. In addition, the ALS patients reported an improvement in Activities of Daily Living/Independence (ADL) and Physical Mobility (PM) sub-scores in the patient self-report health status protocol ALS Assessment Questionnaire 40 (ALSAQ-40). Dextran sulfate treatment further normalized mitochondrial function resulting in an increase in cellular energy state in muscle.

### BRIEF DESCRIPTION OF THE DRAWINGS

The embodiments, together with further objects and advantages thereof, may best be understood by making reference to the following description taken together with the accompanying drawings, in which:

FIG. 1 schematically illustrates muscle denervation in pathological processes.

FIG. 2 schematically illustrates serum lactate levels in ALS patients after dextran sulfate treatment.

FIG. 3 schematically illustrates ALSAQ-40 ADL scores in ALS patients after dextran sulfate treatment.

FIG. 4 schematically illustrates serum myoglobin levels in ALS patients after dextran sulfate treatment.

FIG. 5 schematically illustrates serum creatine kinase levels in ALS patients after dextran sulfate treatment.

FIG. 6 schematically illustrates serum hepatocyte growth factor (HGF) levels in ALS patients after dextran sulfate treatment.

FIG. 7 serum NAA levels in patients after dextran sulfate treatment.

FIG. 8 serum uric acid levels in patients after dextran sulfate treatment.

FIG. 9 sum of oxypurines in serum in patients after dextran sulfate treatment.

FIG. 10 serum nitrate levels in patients after dextran sulfate treatment.

FIG. 11 serum nitrate+nitrite levels in patients after dextran sulfate treatment.

FIG. 12 serum MDA levels in patients after dextran sulfate treatment.

FIG. 13 serum ALA levels in patients after dextran sulfate treatment.

FIG. 14 serum CITR levels in patients after dextran sulfate treatment.

FIG. 15 serum ORN/CITR levels in patients after dextran sulfate treatment.

FIG. 16 serum α-tocopherol levels in patients after dextran sulfate treatment.

FIG. 17 serum γ-tocopherol levels in patients after dextran sulfate treatment.

### DETAILED DESCRIPTION

The present invention generally relates to muscle atrophy, and treatment or prevention thereof using dextran sulfate, or a pharmaceutically acceptable salt thereof.

Muscle atrophy is the loss of skeletal muscle mass. Common causes of muscle atrophy include immobility, aging, malnutrition, and medications. Current treatment depends on the underlying cause but will often include exercise and adequate nutrition in particular for muscle atrophy caused by immobility, aging or malnutrition (sarcopenia). Muscle atrophy may also be caused by diseases or injuries to the neuromuscular system. For instances, diseases of the muscles linked to genetics, such as muscular dystrophy or myopathies, and denervating conditions like the motor neuron diseases and other neuropathies, can cause atrophy, as well as acute damages to or diseases affecting the nervous system, such as in spinal cord injury or stroke.

The hallmark sign of muscle atrophy is loss of lean muscle mass and symptoms include increased weakness or fasciculation, which may result in difficulty or inability in performing physical tasks.

Currently, there are no effective medical treatments of muscle atrophy caused by diseases or damages to the neuromuscular system or sarcopenia.

An aspect of the invention relates to dextran sulfate, or a pharmaceutically acceptable salt thereof, for use in treatment or prevention of muscle atrophy in a subject suffering from sarcopenia.

Experimental data as presented herein shows that dextran sulfate treatment arrested muscle atrophy and improved muscle function in patients suffering from amyotrophic lateral sclerosis (ALS). The results of the dextran sulfate treatment included enhanced physical activity of the ALS patients. Furthermore, dextran sulfate did not only reduce muscle degeneration as seen in a significant reduction in serum creatine kinase and myoglobin but also improved muscle function and activity as seen in significantly raised levels of serum lactate. In addition, the ALS patients reported an improvement in Activities of Daily Living/Independence (ADL) and Physical Mobility (PM) subscores in the patient self-report health status protocol ALS Assessment Questionnaire 40 (ALSAQ-40). A high serum concentration of alanine (ALA) is caused by higher rate of muscular protein degradation in ALS patients. Dextran sulfate treatment normalized circulating values of ALA indicating a positive action of dextran sulfate on muscle metabolism and functions.

Muscle atrophy, particularly sarcopenia, is characterized by energy metabolism imbalance and impaired mitochondrial function leading to the activation of the adenine nucleotide degradation pathway with a consequent increase in circulating purine compounds. Dextran sulfate treatment decreased serum concentrations of uric acid and sum of oxypurines, indicating amelioration of mitochondrial functions with increase in cellular energy state in muscles.

Additionally, dextran sulfate treatment had the effect of significantly lowering serum N-acetylaspartate (NAA) concentrations indicating a protective effect on neuron survival. Dextran sulfate treatment further decreased serum concentrations of nitrate, nitrite+nitrate and malondialdehyde (MDA) indicating a positive influence on mitochondrial function ultimately causing a lowering of reactive oxygen species (ROS) production. Dextran sulfate further reduced nitrosative stress as seen by induction of lower serum concentrations of citruline (CITR) and higher values of the ornithine (ORN)/CITR ratio.

Hence, dextran sulfate of the embodiments induced various positive effects effective in treating or preventing muscle atrophy, and in particular treating or preventing muscle atrophy in subjects suffering from sarcopenia.

Sarcopenia is a type of muscle atrophy that occurs with aging and/or immobility. It is characterized by the degenerative loss of skeletal muscle mass, quality, and strength. The rate of muscle loss is dependent on exercise level, co-morbidities, nutrition and other factors. The muscle loss is related to changes in muscle synthesis signaling pathways. It is distinct from cachexia, in which muscle is degraded through cytokine-mediated degradation, although both conditions may co-exist.

The effects of dextran sulfate treatment as summarized above and further described in the Example section are thereby effective in counteracting the degenerative loss of skeletal muscle mass, quality and strength associated with muscle atrophy in subjects suffering from sarcopenia.

Dextran sulfate, or a pharmaceutically acceptable salt thereof, is therefore suitable for use in treatment or prevention of sarcopenia.

Dextran sulfate, or the pharmaceutically acceptable salt thereof, is in particular useful in treatment or prevention of muscle atrophy of skeletal muscle.

Another aspect of the invention relates to dextran sulfate, or the pharmaceutically acceptable salt thereof, for use in improving muscle function in a subject suffering from a neuromuscular disease and/or damage or sarcopenia.

In an embodiment, the subject is suffering from a neuromuscular disease and/or damage.

The neuromuscular disease could either be an acute condition, i.e., an acute neuromuscular disease or condition, or a chronic condition, i.e., a chronic neuromuscular disease or condition.

Neuromuscular diseases and/or damages include various diseases and disorders and damages, injuries or insults that impair the functioning of muscles. Such diseases and/or damages may either directly affect muscles, i.e., being pathologies of the voluntary muscle, or indirectly affecting muscles, such as being pathologies of nerves or neuromuscular junctions.

Hence, in an embodiment, the subject is suffering from an intrinsic muscle disease and/or damage.

Illustrative examples of such intrinsic muscle diseases include muscular dystrophy (MD), Duchenne muscular dystrophy (DMD), ALS and myositis.

MD is a group of muscle diseases that results in increasing weakening and breakdown of skeletal muscles over time. The diseases differ, in which muscles are primarily affected, the degree of weakness, how fast they worsen, and when symptoms begin. The muscular dystrophy group contains thirty different genetic disorders that are usually classified into nine main categories or types. The most common type is DMD. Other types include Becker muscular dystrophy (BMD), congenital muscular dystrophy, distal muscular dystrophy, Emery-Dreifuss muscular dystrophy, facioscapulohumeral muscular dystrophy (FSHD), limb-girdle muscular dystrophy (LGMD), myotonic dystrophy and oculopharyngeal muscular dystrophy (OPMD).

DMD is a severe type of muscular dystrophy that primarily affects boys. Muscle weakness usually begins around the age of four, and worsens quickly. Muscle loss typically occurs first in the thighs and pelvis followed by the arms. The disorder is X-linked recessive. It is caused by a mutation in the gene for the protein dystrophin. Dystrophin is important to maintain the muscle fiber's cell membrane.

BMD is an X-linked recessive inherited disorder characterized by slowly progressing muscle weakness of the legs and pelvis. It is a type of dystrophinopathy. This is caused by mutations in the dystrophin gene, which encodes the protein dystrophin. BMD is related to DMD in that both result from a mutation in the dystrophin gene, but has a milder course.

Congenital muscular dystrophy is a group autosomal recessively-inherited muscle diseases characterized by muscle weakness, which is present at birth and the different changes on muscle biopsy that ranges from myopathic to overtly dystrophic due to the age at which the biopsy takes place.

Distal muscular dystrophy is a group of disorders characterized by onset in the hands or feet. Many types involve dysferlin. Miyoshi myopathy, one of the distal muscular dystrophies, causes initial weakness in the calf muscles, and is caused by defects in the same gene responsible for one form of limb-girdle muscular dystrophy.

Emery-Dreifuss muscular dystrophy is a condition that mainly affects muscles used for movement, such as skeletal muscles and also affects the cardiac muscle. Clinical signs include muscle weakness and wasting, starting in the distal limb muscles and progressing to involve the limb-girdle muscles.

FSHD is a type of muscular dystrophy that preferentially weakens the skeletal muscles of the face, those that position the scapula, and those in the upper arm, overlying the humerus bone. FSHD is caused by complex genetic changes involving the DUX4 gene. In those without FSHD, DUX4 is expressed in early human development and later repressed in mature tissues. In FSHD, DUX4 is inadequately turned off, which can be caused by several different mutations, the most common being deletion of DNA in the region surrounding DUX4. This mutation is termed “D4Z4 contraction” and defines FSHD type 1 (FSHD1), making up 95% of FSHD cases. FSHD due to other mutations is classified as FSHD type 2 (FSHD2).

LGMD is a genetically and clinically heterogeneous group of rare muscular dystrophies. It is characterized by progressive muscle wasting which affects predominantly hip and shoulder muscles. LGMD has an autosomal pattern of inheritance.

Myotonic dystrophy is a long-term genetic disorder that affects muscle function. Symptoms include gradually worsening muscle loss and weakness. Muscles often contract and are unable to relax.

Myotonic dystrophy is an autosomal dominant disorder. There are two main types: type 1 (DM1), due to mutations in the DMPK gene, and type 2 (DM2), due to mutations in the CNBP gene.

OPMD is a rare form of muscular dystrophy with symptoms generally starting when an individual is 40 to 50 years old. It can be autosomal dominant neuromuscular disease or autosomal recessive. Symptoms affect muscles of eyelids, face, and throat followed by pelvic and shoulder muscle weakness; it has been attributed to a short repeat expansion in the genome, which regulates the translation of some genes into functional proteins.

ALS, also referred to as Lou Gehrig's disease, is a debilitating disease with varied etiology characterized by rapidly progressive weakness, muscle atrophy and fasciculations, muscle spasticity, dysarthria, dysphagia and dyspnea. ALS is the most common of the motor neuron diseases (ALS, hereditary spastic paraplegia (HSP), primary lateral sclerosis (PLS), progressive muscular atrophy (PMA), progressive bulbar palsy (PBP) and pseudobulbar palsy). The principle characteristic in the pathology of ALS is loss of motor nerve cells in the anterior horns of the spinal cord and in the motor nuclei of the brain stem. This results in secondary atrophy of the corresponding muscles (amyotrophy). Neuroinflammation is a pathological hallmark of ALS and is characterized by activated microglia and infiltrating T cells at sites of neuronal injury. “Lateral sclerosis” refers to corticospinal tract degeneration (lateral in location in the spinal cord). In fact, myelin loss occurs in the corticospinal tract. The sclerosis of ALS, the hardening, involves the lateral columns, or corticospinal tracts and is a secondary phenomenon.

In an embodiment, the subject is suffering from a central and/or peripheral nervous system disease and/or damage.

In an embodiment, the central and/or peripheral nervous system disease and/or damage is selected from the group consisting of stroke, spinal cord injury, traumatic brain injury (TBI), cerebral palsy, Charcot-Marie-Tooth disease (CMT), primary lateral sclerosis (PLS), spinal muscular atrophy (SMA), nerve entrapment, and a surgical complication.

Damage to neurons in the brain or spinal cord can cause prominent muscle atrophy. This can be localized muscle atrophy and weakness or paralysis, such as in cerebrovascular accidents, stroke or spinal cord injury. More widespread damage such as in TBI or cerebral palsy can cause generalized muscle atrophy.

TBI, also known as an intracranial injury, is an injury to the brain caused by an external force. TBI can be classified based on severity, ranging from mild to severe TBI; mechanism, closed or penetrating head injury; or other features, e.g., occurring in a specific location or over a widespread area.

CMT is one of the hereditary motor and sensory neuropathies, a group of varied inherited disorders of the peripheral nervous system characterized by progressive loss of muscle tissue and touch sensation across various parts of the body. CMT was previously classified as a subtype of muscular dystrophy.

PLS is a rare neuromuscular disease with slowly progressive weakness in voluntary muscle movement. PLS affects the upper motor neurons, also called corticospinal neurons, in the arms, legs, and face.

SMA, also called autosomal recessive proximal spinal muscular atrophy and 5q spinal muscular atrophy, is a rare neuromuscular disorder characterized by loss of motor neurons and progressive muscle wasting, often leading to early death. The disorder is caused by a genetic defect in the SMN1 gene, which encodes SMN, a protein widely expressed in all eukaryotic cells and necessary for survival of motor neurons. Lower levels of the protein result in loss of function of neuronal cells in the anterior horn of the spinal cord and subsequent system-wide atrophy of skeletal muscles.

In an embodiment, the subject is suffering from sarcopenia, a disease of distinct etiology from genetic and neural myopathies.

In an embodiment, dextran sulfate, or the pharmaceutically acceptable salt thereof, is formulated for systemic administration to the subject. In an embodiment, dextran sulfate, or the pharmaceutically acceptable salt thereof, is formulated for parenteral administration as an example of systemic administration.

Examples of parenteral administration routes include intravenous (i.v.) administration, intra-arterial administration, intra-muscular administration, intracerebral administration, intracerebroventricular administration, intrathecal administration and subcutaneous (s.c.) administration.

In an embodiment, dextran sulfate, or the pharmaceutically acceptable salt thereof, is preferably formulated for intravenous (i.v.) or subcutaneous (s.c.) administration to the subject. Accordingly, i.v. and s.c. administration are preferred examples of systemic administration of dextran sulfate, or the pharmaceutically acceptable salt thereof. In a particular embodiment, dextran sulfate, or the pharmaceutically acceptable salt thereof, is formulated for s.c. administration to the subject.

In an embodiment, dextran sulfate, or the pharmaceutically acceptable salt thereof, is formulated as an aqueous injection solution, preferably as an aqueous i.v. or s.c. injection solution. Thus, dextran sulfate, or the pharmaceutically acceptable salt thereof, of the embodiments is preferably formulated as an aqueous injection solution with a selected solvent or excipient. The solvent is advantageously an aqueous solvent and in particular a buffer solution. A non-limiting example of such a buffer solution is a citric acid buffer, such as citric acid monohydrate (CAM) buffer, or a phosphate buffer. For instance, dextran sulfate of the embodiments can be dissolved in saline, such as 0.9% NaCl saline, and then optionally buffered with 75 mM CAM and adjusting the pH to about 5.9 using sodium hydroxide. Also non-buffered solutions are possible, including aqueous injection solutions, such as saline, i.e., NaCl (aq). Furthermore, other buffer systems than CAM and phosphate buffers could be used if a buffered solution is desired.

The embodiments are not limited to injections and other administration routes can alternatively be used including nasal, buccal, dermal, tracheal, bronchial, or topical administration. Also, local administration of dextran sulfate, or the pharmaceutically acceptable salt thereof, is possible, such as intramuscular administration.

The active compound, dextran sulfate, is then formulated with a suitable excipient, solvent or carrier that is selected based on the particular administration route.

Carrier refers to a substance that serves as a vehicle for improving the efficiency of delivery and/or the effectiveness of dextran sulfate, or the pharmaceutically acceptable salt thereof.

Excipient refers to a pharmacologically inactive substance that is formulated in combination with dextran sulfate, or the pharmaceutically acceptable salt thereof, and includes, for instance, bulking agents, fillers, diluents and products used for facilitating drug absorption or solubility or for other pharmacokinetic considerations.

Pharmaceutically acceptable salt of dextran sulfate refers to a salt of dextran sulfate having the effects as disclosed herein and not being deleterious to the recipient thereof at the administered dose(s).

Dextran sulfate is preferably a so-called low molecular weight dextran sulfate.

In the following, reference to (average) molecular weight and sulfur content of dextran sulfate applies also to any pharmaceutically acceptable salt of dextran sulfate. Hence, the pharmaceutically acceptable salt of dextran sulfate preferably has the average molecular weight and sulfur content as discussed in the following embodiments.

Dextran sulfate is a sulfated polysaccharide and in particular a sulfated glucan, i.e., a polysaccharide made of many glucose molecules. Average molecular weight as defined herein indicates that individual sulfated polysaccharides may have a molecular weight different from this average molecular weight but that the average molecular weight represents the mean molecular weight of the sulfated polysaccharides. This further implies that there will be a natural distribution of molecular weights around this average molecular weight for a dextran sulfate sample.

Average molecular weight (M_{w}) of dextran sulfate is typically determined using indirect methods, such as gel exclusion/penetration chromatography, light scattering or viscosity. Determination of average molecular weight using such indirect methods will depend on a number of factors, including choice of column and eluent, flow rate, calibration procedures, etc.

Average molecular weight (M_{w}):

$$\frac{\sum {M}_{i}^{2} {N}_{i}}{\sum {M}_{i} {N}_{i}} ,$$

typical for methods sensitive to molecular size rather than numerical value, e.g., light scattering and size exclusion chromatography (SEC) methods. If a normal distribution is assumed, then a same weight on each side of M_{w}, i.e., the total weight of dextran sulfate molecules in the sample having a molecular weight below M_{w} is equal to the total weight of dextran sulfate molecules in the sample having a molecular weight above M_{w}.

In an embodiment, dextran sulfate, or the pharmaceutically acceptable salt thereof, preferably has an average molecular weight equal to or below 40 000 Da, more preferably equal to or below 20 000 Da and in particular equal to or below 10 000 Da.

Dextran sulfate of an average molecular weight exceeding 10 000 Da generally has a lower effect vs. toxicity profile as compared to dextran sulfate having a lower average molecular weight. This means that the maximum dose of dextran sulfate that can be safely administered to a subject is lower for larger dextran sulfate molecules (>10 000 Da) as compared to dextran sulfate molecules having an average molecular weight within the preferred range. As a consequence, such larger dextran sulfate molecules are less appropriate in clinical uses when the dextran sulfate is to be administered to subjects in vivo.

In an embodiment, dextran sulfate, or the pharmaceutically acceptable salt thereof, has an average molecular weight within a range of from 2 000 to 10 000 Da. In another embodiment, the average molecular weight is within a range of from 2 500 to 10 000 Da. In a particular preferred embodiment, the average molecular weight is within a range of from 3 000 to 10 000 Da.

In an optional, but preferred embodiment, less than 40% of the dextran sulfate molecules have a molecular weight below 3 000 Da, preferably less than 35%, such as less than 30% or less than 25% of the dextran sulfate molecules have a molecular weight below 3 000 Da. In addition, or alternatively, less than 20% of the dextran sulfate molecules have a molecular weight above 10 000 Da, preferably less than 15%, such as less than 10% or less than 5% of the dextran sulfate molecules have a molecular weight above 10 000 Da. Thus, in a particular embodiment, the dextran sulfate has a substantially narrow molecular weight distribution around the average molecular weight.

In a particular embodiment, the average molecular weight of dextran sulfate, or the pharmaceutically acceptable salt thereof, is within a range of from 3 500 to 9 500 Da, such as within a range of from 3 500 to 8 000 Da.

In another particular embodiment, the average molecular weight of dextran sulfate, or the pharmaceutically acceptable salt thereof, is within a range of from 4 500 to 7 500 Da.

In a further particular embodiment, the average molecular weight of dextran sulfate, or the pharmaceutically acceptable salt thereof, is within a range of from 4 500 to 5 500 Da.

Thus, in a currently preferred embodiment the average molecular weight of dextran sulfate, or the pharmaceutically acceptable salt thereof, is preferably approximately 5 000 Da or at least substantially close to 5 000 Da, such as 5 000±500 Da, for instance 5 000±400 Da, preferably 5 000±300 Da or 5 000±200 Da, such as 5 000±100 Da. Hence, in an embodiment, the average molecular weight of dextran sulfate, or the pharmaceutically acceptable salt thereof, is 4.5 kDa, 4.6 kDa, 4.7 kDa, 4.8 kDa, 4.9 kDa, 5.0 kDa, 5.1 kDa, 5.2 kDa, 5.3 kDa, 5.4 kDa or 5.5 kDa.

In a particular embodiment, the average molecular weight of dextran sulfate, or the pharmaceutically salt thereof as presented above is average M_{w}, and preferably determined by gel exclusion/penetration chromatography, size exclusion chromatography, light scattering or viscosity-based methods.

Dextran sulfate is a polyanionic derivate of dextran and contains sulfur. The average sulfur content for dextran sulfate of the embodiments is preferably from 15 to 20% and more preferably approximately 17%, generally corresponding to about or at least two sulfate groups per glucosyl residue. In a particular embodiment, the sulfur content of dextran sulfate is preferably equal to or at least close to the maximum possible degree of sulfur content of the corresponding dextran molecules.

In a particular embodiment, dextran sulfate of the embodiments has a number average molecular weight (Mₙ) as measured by nuclear magnetic resonance (NMR) spectroscopy within a range of from 1850 to 3500 Da.

Number average molecular weight (Mₙ):

$$\frac{\sum {M}_{i} {N}_{i}}{\sum {N}_{i}} ,$$

typically derived by end group assays, e.g., NMR spectroscopy or chromatography. If a normal distribution is assumed, then a same number of dextran sulfate molecules can be found on each side of Mₙ, i.e., the number of dextran sulfate molecules in the sample having a molecular weight below Mₙ is equal to the number of dextran sulfate molecules in the sample having a molecular weight above Mₙ.

In a preferred embodiment, dextran sulfate of the embodiments has a Mₙ as measured by NMR spectroscopy within a range of from 1850 to 2500 Da, preferably within a range of from 1850 to 2300 Da, and more preferably within a range of from 1850 to 2000 Da.

In a particular embodiment, dextran sulfate of the embodiments has an average sulfate number per glucose unit within a range of from 2.5 to 3.0, preferably within a range of from 2.5 to 2.8, and more preferably within a range of from 2.6 to 2.7.

In a particular embodiment, dextran sulfate of the embodiments has an average number of glucose units within a range of from 4.0 to 6.0, preferably within a range of from 4.5 to 5.5, and more preferably within a range of from 5.0 to 5.2, such as about 5.1.

In another particular embodiment, dextran sulfate of the embodiments has on average 5.1 glucose units and an average sulfate number per glucose unit of 2.6 to 2.7, typically resulting in a number average molecular weight (Mₙ) as measured by NMR spectroscopy within a range of from 1850 to 2000 Da.

A dextran sulfate, or pharmaceutically salt thereof, that can be used according to the embodiments is described in WO 2016/076780.

The dextran sulfate according to the embodiments can be provided as a pharmaceutically acceptable salt of dextran sulfate. Such pharmaceutically acceptable salts include e.g., a sodium or potassium salt of dextran sulfate. In a particular embodiment, the pharmaceutically acceptable salt is a sodium salt of dextran sulfate.

In a particular embodiment, the sodium salt of dextran sulfate, including Na⁺ counter ions, has a Mₙ as measured by NMR spectroscopy within a range of from 2000 to 2500 Da, preferably within a range of 2100 and 2300 Da.

In an embodiment, an effective amount of dextran sulfate, or the pharmaceutically acceptable salt thereof, is administered to the subject. Effective amount as used herein relates to a therapeutically effective amount of dextran sulfate, or the pharmaceutically acceptable salt thereof, capable of causing a medical effect when administered to the subject that is related to an improvement of the muscle function and status of the subject. Such a therapeutically effective amount is preferably an amount of dextran sulfate, or the pharmaceutically acceptable salt thereof, capable of inducing a change in at least one biomarker associated with muscle function, such as serum creatine kinase or myoglobin. The therapeutically effective amount of dextran sulfate, or the pharmaceutically salt thereof, can be determined by the physician and may, optionally, be selected based on at least one among the sex of the subject, the weight of the subject, the age of the subject, the type of neuromuscular disease or damage and the severity of the neuromuscular disease or damage.

Suitable dose ranges for the dextran sulfate, or the pharmaceutically acceptable salt thereof, of the embodiments may vary according to the size and weight of the subject, the condition for which the subject is treated, and other considerations. In particular for human subjects, a possible dosage range could be from 1 μg/kg to 150 mg/kg of body weight, preferably from 10 μg/kg to 100 mg/kg of body weight.

In preferred embodiments, dextran sulfate, or the pharmaceutically acceptable salt thereof, is formulated to be administered at a dosage in a range from 0.05 to 50 mg/kg of body weight of the subject, preferably from 0.05 or 0.1 to 40 mg/kg of body weight of the subject, and more preferably from 0.05 or 0.1 to 30 mg/kg, or 0.1 to 25 mg/kg or from 0.1 to 15 mg/kg or 0.1 to 10 mg/kg body weight of the subject. A currently preferred dosage of dextran sulfate, or the pharmaceutically acceptable salt thereof, is from 0.5 to 5 mg/kg body weight of the subject.

Administration of dextran sulfate, or the pharmaceutically acceptable salt thereof, does not necessarily have to be limited to treatment of a muscle atrophy but could alternatively, or in addition, be used for prophylaxis. In other words, dextran sulfate of the embodiments could be administered to a subject having an increased risk of developing muscle atrophy.

Treatment of muscle atrophy also encompasses inhibition of muscle atrophy. Inhibition of muscle atrophy as used herein implies that dextran sulfate, or the pharmaceutically acceptable salt thereof, reduces the symptoms and effects of the condition even though a 100% treatment or cure does not necessarily occur. For instance, inhibition of muscle atrophy may involve an improvement in muscle function.

Dextran sulfate, or the pharmaceutically acceptable salt thereof, of the embodiments can be administered at a single administration occasion, such as in the form of a single injection or bolus injection. This bolus dose can be injected quite quickly to the subject but is advantageously infused over time so that the dextran sulfate solution is infused over a few minutes of time to the subject, such as during 5 to 10 minutes or more. Also slow-release formulations of dextran sulfate, or the pharmaceutically acceptable salt thereof, of the embodiments can be used in order to achieve a prolonged release thereof.

Alternatively, dextran sulfate, or the pharmaceutically acceptable salt thereof, of the embodiments can be administered at multiple, i.e., at least two, occasions during a treatment period. Thus, dextran sulfate of the embodiments could be administered once or at multiple times per day, once or at multiple times per week, once or at multiple times per month as illustrative examples.

In a particular embodiment, dextran sulfate, or the pharmaceutically acceptable salt thereof, is formulated for administration at 1-14 times, preferably 1-7 times, a week for one or multiple consecutive weeks, such as at least 2-5 consecutive weeks. In a particular embodiment, dextran sulfate, or the pharmaceutically acceptable salt thereof, is formulated for administration once or twice a day for multiple days, such as multiple consecutive days, e.g., 2-14 days.

It is also possible combine a bolus injection of dextran sulfate, or the pharmaceutically acceptable salt thereof, with one or more additional administrations of dextran sulfate, or the pharmaceutically acceptable salt thereof.

In an embodiment, the subject is a mammalian subject, preferably a primate, and more preferably a human subject. Although the embodiments are in particular directed towards treatment of muscle atrophy in human subjects, the embodiments may also, or alternatively, be used in veterinary applications. Non-limiting example of animal subjects include non-human primate, cat, dog, pig, horse, mouse, rat, goat, guinea pig, sheep and cow.

The invention also relates to the use of dextran sulfate, or a pharmaceutically acceptable salt thereof, in the manufacture of a medicament for treatment or prevention of muscle atrophy in a subject suffering from sarcopenia or improving muscle function in a subject suffering from a neuromuscular disease and/or damage or sarcopenia.

The invention also defines a method for treating or preventing muscle atrophy. The method comprises administering dextran sulfate, or a pharmaceutically acceptable salt thereof, to a subject suffering from sarcopenia to treat or prevent muscle atrophy. The invention further defines a method of improving muscle function. The method comprises administering dextran sulfate, or the pharmaceutically acceptable salt thereof, to a subject suffering from a neuromuscular disease and/or damage or sarcopenia to improve muscle function in the subject.

### EXAMPLES

In ALS, both the upper motor neurons and the lower motor neurons degenerate or die and stop sending messages to the muscles. Unable to function, the denervating muscles gradually weaken, start to twitch (called fasciculations), and waste away (atrophy). Eventually, the brain loses its ability to initiate and control voluntary movements. Gradually all muscles under voluntary control are affected, and individuals lose their strength and the ability to speak, eat, move, and even breathe.

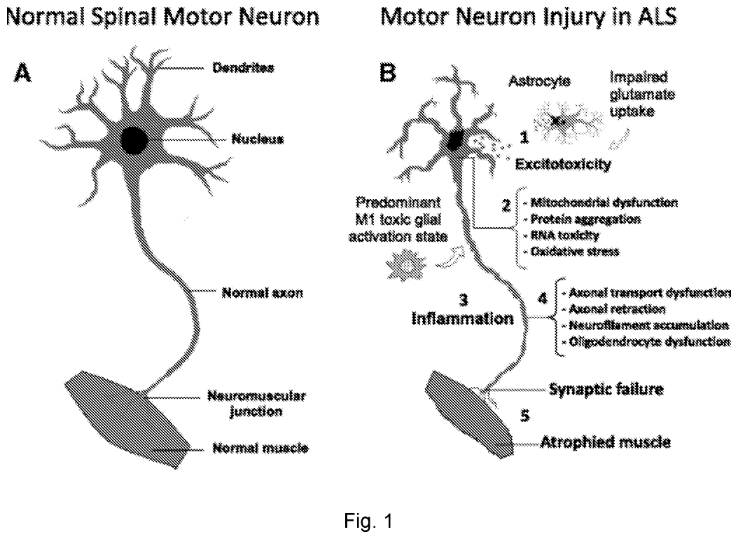

Muscle atrophy and impaired physical activity is an early symptom of ALS, reflecting the muscle denervation that is characteristic of the disease. The muscle denervation is a consequence of several pathological processes, see FIG. 1:
1) Astrocytes are not able to support neuronal functions and impaired glutamate clearance leads to neuronal excitotoxicity;
2) Defects in protein degradation pathways and disturbances in RNA processing result in protein aggregate formation, RNA toxicity and mitochondrial dysfunction;
3) The secretion of pro-inflammatory cytokines by predominant M1 activated microglia contributes to the development of an inflammatory milieu; and
4) Failure of axonal architecture and transport functions, together with the alteration of the physiological role of oligodendrocytes results in 5) synaptic failure, denervation and finally, muscle atrophy.

### Example 1

Using a longitudinal design, this study was aimed to determine the changes of selected serum metabolites in ALS patients before dextran sulfate administration and after different times following the beginning of the treatment. Changes in the measured metabolites indicated the biochemical response of the patient to dextran sulfate that is underpinning potential disease modification and the mechanisms of action of the drug in this ALS patient population.

Materials and Methods

Dextran sulfate (Tikomed AB, Viken, Sweden, WO 2016/076780) was administered at 2 mg/kg by daily subcutaneous injection once a week for ten weeks to 8 human patients suffering from ALS.

Peripheral venous blood samples were collected from patients before (week 0) and after dextran sulfate administration (week 5 and week 10) after at least 15 minutes of complete rest, using the standard tourniquet procedure, from the antecubital vein into a single VACUETTE® polypropylene tube containing serum separator and clot activator (Greiner-Bio One GmbH, Kremsmunster, Austria). After 30 mins at room temperature (20-25° C.), blood withdrawals were centrifuged at 1,890×g for 10 min to get serum aliquots.

A serum aliquot of 500 μl was supplemented with 1 ml of HPLC-grade acetonitrile, vortexed for 60 secs, centrifuged at the maximum speed in a top-bench centrifuge to precipitate proteins. Supernatants were washed with large volumes of HPLC-grade chloroform to remove organic solvent, centrifuged and the upper aqueous phases were transferred to different tubes, clearly labeled to identify the sample and stored at −80° C. until analyzed to determine different water-soluble compounds.

ALSAQ-40 was assessed pre-treatment and weekly thereafter for the 10 treatment weeks and for the visits at the follow-up period.

Results

Lactate is made by muscles and accumulates in the blood during exercise in normal and ALS patients. Raised levels of serum lactate indicate improved muscle function/use.

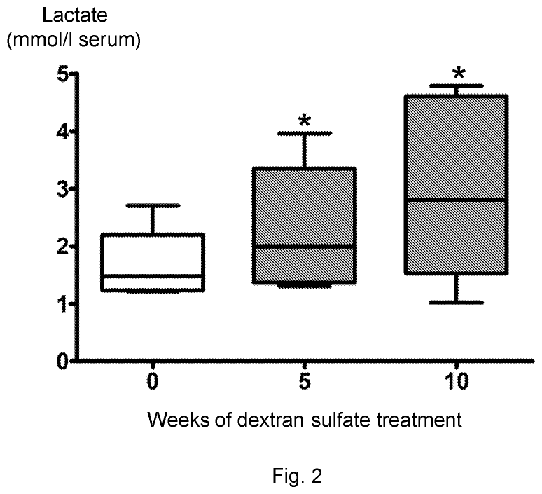

FIG. 2 illustrates serum lactate levels in ALS patient prior to (Week 0) and after dextran sulfate administration. The data shows that the circulating level of lactate, mainly deriving from metabolism of muscle cells, was raised significantly with increasing time of dextran sulfate administration (*significantly different compared to Week 0, p<0.01). After 5 weeks of dextran sulfate treatment, serum lactate levels increased by 29.8% from 1.78±0.59 to 2.31±1.02 μmol/L (p<0.01, Wilcoxon signed-rank test), whilst after 10 weeks of dextran sulfate treatment, serum lactate levels had increased by 70% to 3.02±1.59 μmol/L. Hence, dextran sulfate administration led to increased muscular activity in the ALS patients.

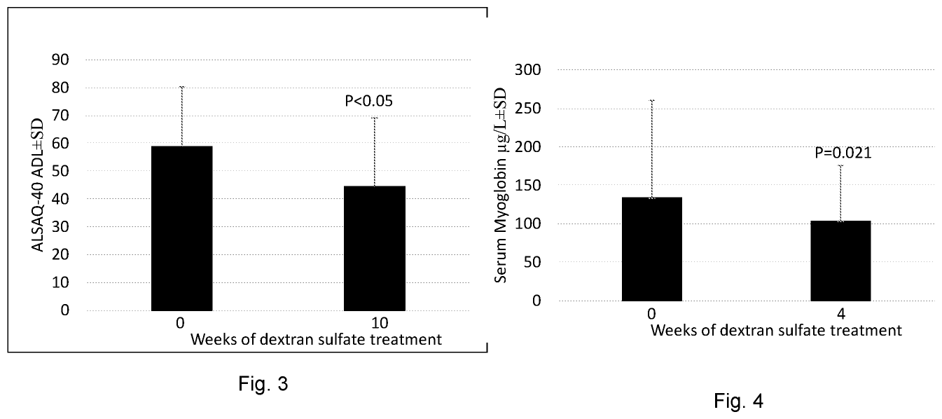

The ALSAQ-40 sub-scores entitled ‘Activities of Daily Living/Independence’ (ADL) and ‘Physical Mobility’ (PM) reflect the patients' view of their level of physical activity and independence. A falling score reflects improved physical activity. After 10 weeks of dextran sulfate treatment the ADL sub-score declined significantly by 18.6% from 58.9±21.4 to 44.4±24.7 (p<0.05), see FIG. 3, whilst the PM sub-score declined by 16% from 27.2±22.2 to 22.7±20.2. These results indicated improved physical activity of the patients during treatment.

### Example 2

A clinical trial in the form of a Phase IIa, single-center, open label, single-arm study where the safety, tolerability and possible efficacy of subcutaneously administered dextran sulfate was evaluated in patients with ALS of intermediate progression rate. The clinical trial was conducted at the Sahlgrenska University Hospital, Gothenburg, Sweden and was overseen and approved by the Ethics Committee of the University of Gothenburg and by the Swedish Medical Products Agency.

Materials and Methods

Dextran sulfate (Tikomed AB, Viken, Sweden, WO 2016/076780) was administered at 1 mg/kg by daily subcutaneous injection once a week for five weeks to 13 human patients suffering from ALS.

Blood samples were drawn at defined study intervals by a venous catheter into vacutainer tubes. Laboratory analyses of blood plasma were performed immediately after collection by the Clinical Chemistry Laboratory at the Sahlgrenska University Hospital.

ALSFRS-R was assessed pre-treatment and weekly thereafter for the 5 treatment weeks and for the visits at the follow-up period.

Results

Myoglobin is a protein typically found in heart and skeletal muscle tissues. A raised level of myoglobin is found in the bloodstream when injury/disease has damaged muscle. Reduced levels of serum myoglobin indicate reduced muscle degeneration.

Serum myoglobin data from the patients revealed a statistically significant 30% reduction in myoglobin levels from 133.92±126.28 to 103.69±72.16 μg/L (p=0.021 vs Day 1) after 4 weeks of dextran sulfate treatment indicating a drug-related reduction in the rate of muscle tissue degeneration and muscle atrophy of the patients during treatment, see FIG. 4.

The appearance in blood of the muscle enzyme creatine kinase is generally considered to be a biomarker of muscle damage, and to be particularly useful for the diagnosis of medical conditions involving muscle atrophy, including ALS. Raised creatine kinase levels are a common characteristic of ALS patients. Reduced levels of serum creatine kinase indicates relief from the disease-related myopathy.

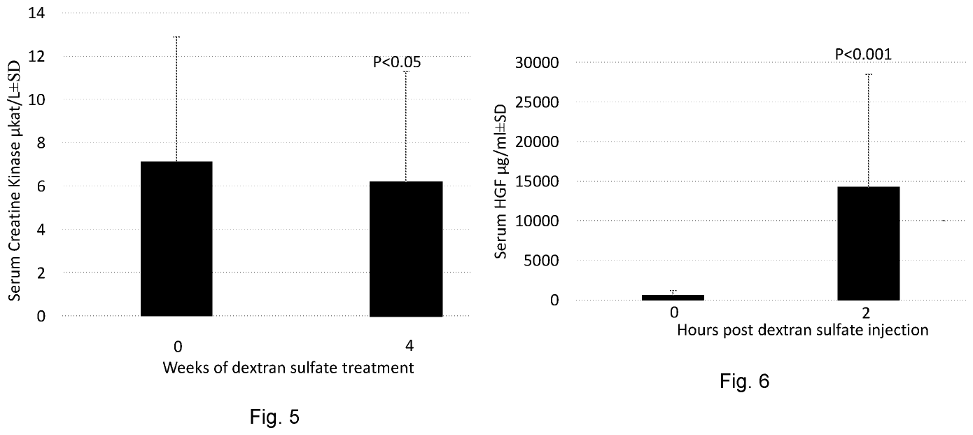

Serum creatine kinase data from the patients revealed a statistically significant 13.3% reduction in levels from 7.15±5.74 to 6.2±5.08 μkat/L (p<0.05, Wilcoxon signed-rank test) after 4 weeks of dextran sulfate treatment, indicating a drug-related reduction in muscle atrophy of the patients during treatment, see FIG. 5.

Hepatocyte growth factor (HGF) is a naturally occurring growth factor that acts as a potent neuroprotective and myogenic agent and has been shown to be useful against degenerative disease progression in numerous animal models, including in ALS models. Interestingly, Hauerslev S et al. (2014, Plos One 9:e100594) demonstrated an 18% increase in muscle mass after 2 weeks of recombinant HGF treatment in a mouse model of muscle atrophy. The observation that HGF treatment is able to induce such rapid regenerative responses in skeletal muscle in this animal model of muscle atrophy is of relevance to the rapid muscle responses observed in response to dextran sulfate in the ALS patients.

Pharmacokinetic data from the ALS patients revealed a statistically significant (p<0.001) increase to pharmacologically relevant levels of circulating HGF from 820±581 to a peak at 2.5 hours of 37863±14235 μg/L after dextran sulfate injection, see FIG. 6. This indicates the potential for direct myogenic, as well as indirect neurotrophic, HGF-mediated effects on muscle atrophy after dextran sulfate administration.

The biochemical evidence of reduced muscle degeneration supports the clinical observations of improved muscle function. For example, in the ALSFRS-R, functions mediated by cervical, trunk, lumbosacral, and respiratory muscles are each assessed by 3 items and scores in these categories show close agreement with objective measures of muscle strength. Of note, two patients with severe bulbar paresis experienced almost complete resolution of this symptom during the five-week treatment period.

### Example 3

The objective of this Example was to evaluate the effect of repeated dextran sulfate administration on the concentrations of serum metabolites in serum samples from a cohort of Swedish ALS patients who had participated in the trial entitled ‘A single-centre, open single-arm study where the safety, tolerability and efficacy of subcutaneously administered ILB® will be evaluated in patients with Amyotrophic Lateral Sclerosis’. Using a longitudinal design, this study aimed to determine the changes of selected serum metabolites in each ALS patient before and after weekly dextran sulfate treatment. Changes in the measured metabolites indicate the biochemical response of the patient to dextran sulfate that is underpinning potential disease modification and the mechanisms of action of the drug in this patient population.

Materials & Methods

After an initial screening visit, patients had five weekly dosing of a single dextran sulfate (ILB®, Tikomed AB, Viken, Sweden, WO 2016/076780) injection of 1.0 mg/kg body weight in saline into the subcutaneous fat of the lower abdomen.

Peripheral venous blood samples were been collected from the patients after at least 15 minutes of complete rest, using the standard tourniquet procedure, from the antecubital vein into a single VACUETTE® polypropylene tube containing serum separator and clot activator (Greiner-Bio One GmbH, Kremsmunster, Austria). After 30 minutes at room temperature, blood withdrawals were centrifuged at 1,890×g for 10 min and the resulting serum samples were stored at −20° C. until analysis.

After defrost, an aliquot of 500 μl of each serum sample was supplemented with 1 ml of HPLC-grade acetonitrile, vortexed for 60 seconds, centrifuged at the maximum speed in a top-bench centrifuge to precipitate proteins. Supernatants were washed with large volumes of HPLC-grade chloroform to remove organic solvent, centrifuged and the upper aqueous phases were transferred in different tubes, clearly labeled to identify the sample and stored at −80° C. until analyzed to determine different water-soluble compounds.

A second aliquot of about 300 μl of each serum sample was light-protected and then processed to extract fat-soluble antioxidants using a method described in detail elsewhere (Lazzarino et al., Single-step preparation of selected biological fluids for the high performance liquid chromatographic analysis of fat-soluble vitamins and antioxidants. J Chromatogr A. 2017; 1527: 43-52.). Briefly, samples were supplemented with 1 ml of HPLC-grade acetonitrile, vigorously vortexed for 60 s and incubated at 37° C. for 1 h in a water bath under agitation, to allow the full extraction of lipid soluble compounds. Samples were then centrifuged at 20,690×g for 15 min at 4 C to precipitate proteins and the clear supernatants were saved at −80° C. until the HPLC analysis of fat-soluble vitamins and antioxidants.

In deproteinized serum samples, the following water-soluble compounds were separated and quantified by HPLC, according to methods described elsewhere (Tavazzi et al., Simultaneous high performance liquid chromatographic separation of purines, pyrimidines, N-acetylated amino acids, and dicarboxylic acids for the chemical diagnosis of inborn errors of metabolism. Clin Biochem. 2005; 38: 997-1008; Romitelli et al., Comparison of nitrite/nitrate concentration in human plasma and serum samples measured by the enzymatic batch Griess assay, ion-pairing HPLC and ion-trap GC-MS: The importance of a correct removal of proteins in the Griess assay. J Chromatogr B Analyt Technol Biomed Life Sci. 2007; 851: 257-267; Amorini et al., Metabolic profile of amniotic fluid as a biochemical tool to screen for inborn errors of metabolism and fetal anomalies. Mol Cell Biochem. 2012; 359: 205-216): hypoxhantine, xanthine, uric acid, malondialdehyde (MDA), nitrite, nitrate, N-acetylaspartate (NAA), citrulline (CITR), alanine (ALA), and ornithine (ORN).

The following fat-soluble vitamins and antioxidants in deproteinized serum samples were separated and quantified by HPLC according to a method as previously described (Lazzarino et al., Cerebrospinal fluid ATP metabolites in multiple sclerosis. Mult Scler J. 2010; 16: 549-554): α-tocopherol (vitamin E) and γ-tocopherol.

Statistics

Comparison of the Pre- and Post-treatment subgroups was performed by the two-tailed Student's t-test for paired samples. The comparison of each subgroup with the group of control healthy subjects was carried out by the two-tailed non-parametric Mann-Whitney U-test for unpaired observations. Differences with p<0.05 were considered statistically significant.

Results

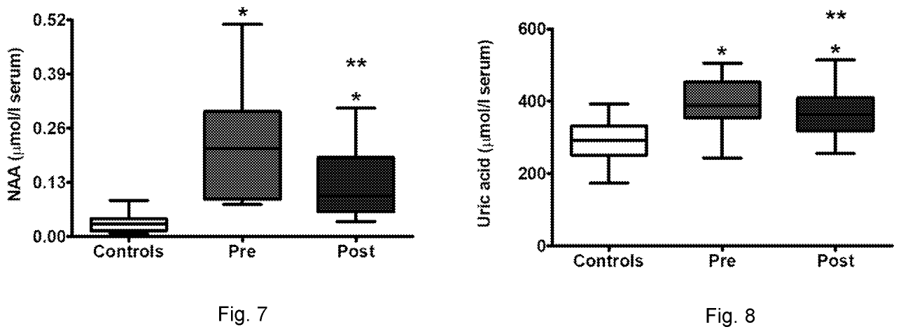

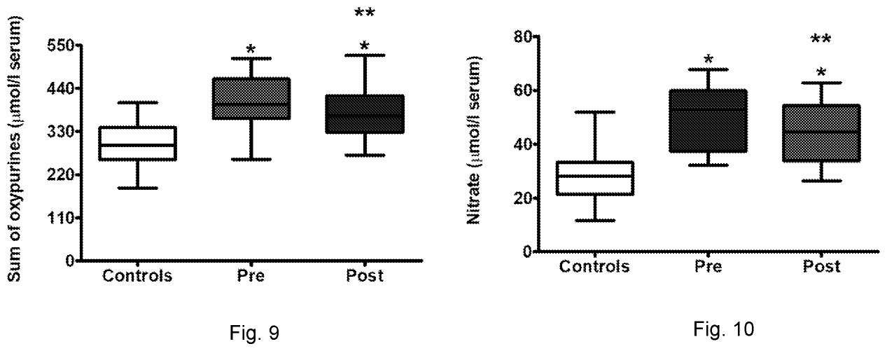

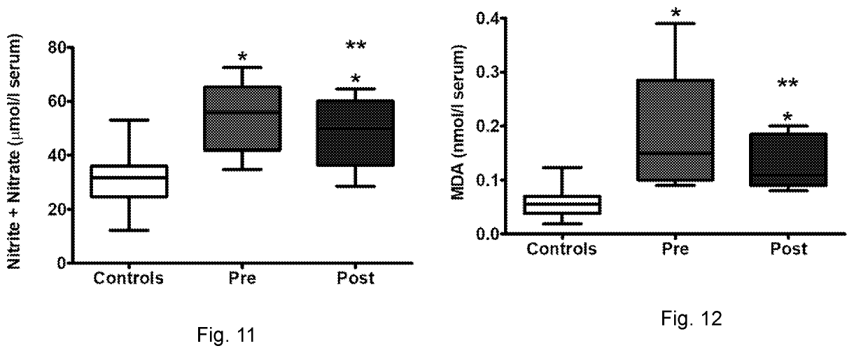

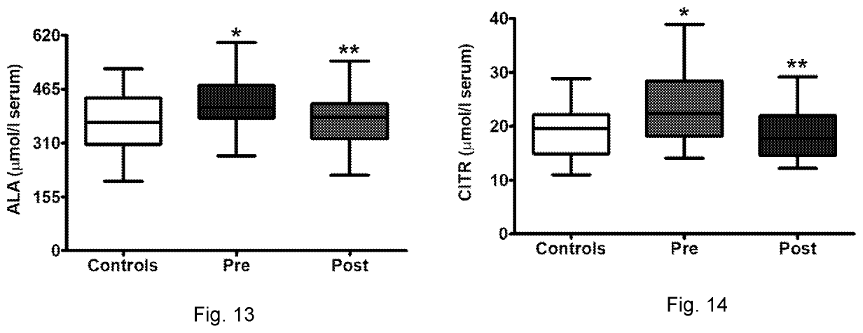

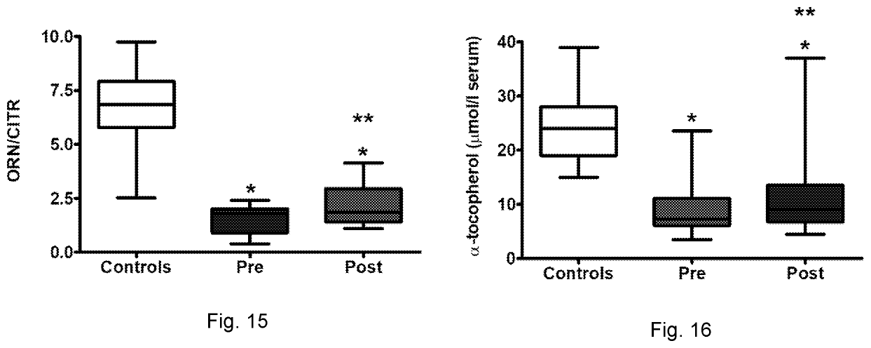

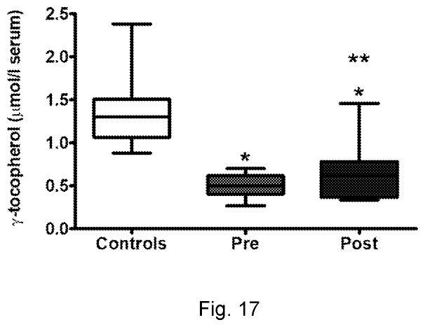

According to the statistical analysis, the two pre- and post-treatment subgroups of patients had significantly different serum concentrations of NAA (FIG. 7), uric acid (FIG. 8), MDA (FIG. 12), nitrate (NO₃) (FIG. 10), nitrite+nitrate (NO₃+NO₂) (FIG. 11), sum oxypurines (FIG. 9), ALA (FIG. 13), CITR (FIG. 14), ORN/CITR ratio (FIG. 15), and vitamin E (α-tocopherol and γ-tocopherol) (FIGS. 16 and 17).

Data of the aforementioned compounds are illustrated into box plots (reporting minimum, maximum, median, 25% and 75% percentiles) in FIGS. 7-17, in which values of control healthy subjects (age ranging 25-65 years), from historical data sets derived from an Italian cohort of patients, have also been included and compared to both groups (pre- and post-treatment) of the patients. In all the figures, differences respect to the value of controls is indicated by one asterisk (*), whilst difference between the two subgroups of patients is indicated by two asterisks (**).

The ALS patients had higher serum NAA levels than those measured in control healthy subjects, presumably deriving from a decrease in viable neurons (FIG. 7). Significantly lower NAA values were measured post-dextran sulfate treatment, thus suggesting a protective effect on cellular survival of the drug.

The ALS patients had higher serum concentrations of uric acid and sum of oxypurines (hypoxanthine+xanthine+uric acid) (FIGS. 8 and 9) than those measured in controls, in consequence of energy metabolism imbalance leading to the activation of the adenine nucleotide degradation pathway with a consequent increase in circulating purine compounds. Dextran sulfate treatment decreased both these parameters, thus suggesting amelioration of mitochondrial functions with increase in cell energy state.

The ALS patients had higher serum concentrations of nitrate (FIG. 10), nitrite+nitrate (FIG. 11) and MDA (FIG. 12) than those measured in controls, strongly indicating sustained oxidative/nitrosative stress, causing increasing in the circulating levels of these stable end-products of ROS-mediated lipid peroxidation (MDA) and of nitric oxide metabolism (nitrate and nitrite+nitrate). Dextran sulfate treatment decreased the levels of these parameters, thus, indicating either a direct scavenging activity of the compound, a positive influence on genes, such as BDNF, regulating the levels of scavenger enzymes, and/or or a positive influence on mitochondrial functions ultimately causing lower levels of ROS production.

The ALS patients had higher serum concentrations of ALA than those measured in controls (FIG. 13), because of a higher rate of muscular protein degradation. Dextran sulfate treatment normalized circulating values of ALA (equal to those of controls and significantly lower than those of the pre-treatment subgroup), suggesting a positive action of the drug on muscle metabolism and functions.

The ALS patients had higher serum concentrations of CITR (FIG. 14) and lower values of the ORN/CITR ratio (FIG. 15) than those measured in controls, thereby corroborating the presence of sustained nitrosative stress caused by excess of nitric oxide production and subsequent increase in reactive nitrogen species (RNS). Dextran sulfate treatment ameliorated both parameters, possibly by lowering the expression of inducible nitric oxide synthase, the enzyme responsible for triggering nitrosative stress.

The ALS patients had lower serum concentrations of both α-tocopherol and γ-tocopherol (the two main forms of Vitamin E) than those measured in controls (FIGS. 16 and 17), thereby suggesting significant decrease of the main fat-soluble antioxidant playing a key role as an interrupter of the ROS-mediated peroxidation of fatty acids of membrane phospholipids. Dextran sulfate treatment ameliorated both parameters.

### Example 4

The effects of daily sub-cutaneous injections of dextran sulfate on glutamate excitotoxicity and mitochondrial function after severe traumatic brain injury (sTBI) in rats were evaluated by high-performance liquid chromatography (HPLC) analysis of frozen brain samples. The results suggest that dextran sulfate interferes with mitochondrial function to improve energy metabolism and also decreases glutamate excitotoxicity.

Materials & Methods

Induction of sTBI and Drug Administration Protocol

The experimental protocol used in this study was approved by the Ethical Committee of the Catholic University of Rome, according to international standards and guidelines for animal care. Male Wistar rats of 300-350 g body weight (b.w.) were fed with standard laboratory diet and water ad libitum in a controlled environment.

They were divided into three groups:
1) n=6 animals subjected to sTBI, with drug administration after 30 minutes and sacrifice at 2 days post-TBI (Acute phase 1)
2) n=6 animals subjected to severe-TBI, with drug administration after 30 minutes and sacrifice at 7 days post-TBI (Acute phase 2).
3) n=6 animals subjected to severe-TBI, with drug administration after 3 days and sacrifice at 7 days post-TBI (Chronic phase).

As the anesthetic mixture, animals received 35 mg/kg b.w. ketamine and 0.25 mg/kg b.w. midazolam by i.p. injection. sTBI was induced by dropping a 450 g weight from 2 m height on to the rat head that had been protected by a metal disk previously fixed on the skull, according to the “weight drop” impact acceleration model (Marmarou et al., A new model of diffuse brain injury in rats. Part I: Pathophysiology and biomechanics. J Neurosurg. 1994; 80: 291-300). Rats that suffered from skull fracture, seizures, nasal bleeding, or did not survive the impacts, were excluded from the study. At the end of each period of treatment, rats were anesthetized again and then immediately sacrificed.

The drug treatment was a subcutaneous injection of 0.5 ml of dextran sulfate (Tikomed AB, Viken, Sweden, WO 2016/076780; 15 mg/kg) and administered according to the aforementioned schematic protocol.

Cerebral Tissue Processing

An in vivo craniectomy was performed in all animals during anesthesia, after carefully removing the rat's skull, the brain was exposed and removed with a surgical spatula and quickly dropped in liquid nitrogen. After the wet weight (w.w.) determination, tissue preparation was affected as previously disclosed (Tavazzi et al., Cerebral oxidative stress and depression of energy metabolism correlate with severity of diffuse brain injury in rats. Neurosurgery. 2005; 56: 582-589; Vagnozzi et al., Temporal window of metabolic brain vulnerability to concussions: mitochondrial-related impairment-part I. Neurosurgery. 2007; 61: 379-388; Tavazzi et al., Temporal window of metabolic brain vulnerability to concussions: oxidative and nitrosative stresses-part II. Neurosurgery. 2007; 61: 390-395; Amorini et al., Severity of experimental traumatic brain injury modulates changes in concentrations of cerebral free amino acids. J Cell Mol Med. 2017; 21: 530-542.). Briefly, whole brain homogenization was performed with 7 ml of ice-cold, nitrogen-saturated, precipitating solution composed by CH₃CN+10 mM KH₂PO₄, pH 7.40, (3:1; v:v), and using an Ultra-Turrax set at 24,000 rpm/min (Janke & Kunkel, Staufen, Germany). After centrifugation at 20,690×g, for 10 min at 4° C., the clear supernatants were saved, pellets were supplemented with 3 ml of the precipitating solution and homogenized again as described above. A second centrifugation was performed (20,690×g, for 10 min at 4° C.), pellets were saved, supernatants combined with those previously obtained, extracted by vigorous agitation with a double volume of HPLC-grade CHCl₃ and centrifuged as above. The upper aqueous phases containing water-soluble low-molecular weight compounds were collected, subjected to chloroform washings for two more times (this procedure allowed the removal of all the organic solvent and of any lipid soluble compound from the buffered tissue extracts), adjusted in volumes with 10 mM KH₂PO₄, pH 7.40, to have ultimately aqueous 10% tissue homogenates and saved at −80° C. until assayed.

HPLC Analyses of Purine-Pyrimidine Metabolites

Aliquots of each deproteinized tissue samples were filtered through a 0.45 μm HV Millipore filter and loaded (200 μl) onto a Hypersil C-18, 250×4.6 mm, 5 μm particle size column, provided with its own guard column (Thermo Fisher Scientific, Rodano, Milan, Italy) and connected to an HPLC apparatus consisting of a Surveyor System (Thermo Fisher Scientific, Rodano, Milan, Italy) with a highly sensitive diode array detector (equipped with a 5 cm light path flow cell) and set up between 200 and 300 nm wavelength. Data acquisition and analysis were performed by a PC using the ChromQuest® software package provided by the HPLC manufacturer.

Metabolites belonging to the purine-pyrimidine profiles (listed below) and related to tissue energy state, mitochondrial function and relative to oxidative-nitrosative stresses were separated, in a single chromatographic run, according to slight modifications of existing ion-pairing HPLC methods (Lazzarino et al., Single-sample preparation for simultaneous cellular redox and energy state determination. Anal Biochem. 2003; 322: 51-59; Tavazzi et al., Simultaneous high performance liquid chromatographic separation of purines, pyrimidines, N-acetylated amino acids, and dicarboxylic acids for the chemical diagnosis of inborn errors of metabolism. Clin Biochem. 2005; 38: 997-1008). Assignment and calculation of the compounds of interest in chromatographic runs of tissue extracts were carried out at the proper wavelengths (206, 234 and 260 nm) by comparing retention times, absorption spectra and areas of peaks with those of peaks of chromatographic runs of freshly-prepared ultra-pure standard mixtures with known concentrations.

List of compounds: Cytosine, Creatinine, Uracil, Beta-Pseudouridine, Cytidine, Hypoxanthine, Guanine, Xanthine, Cytidine diphosphate-Choline (CDP-Choline), Ascorbic Acid, Uridine, Adenine, Nitrite (—NO₂⁻), reduced glutathione (GSH), Inosine, Uric Acid, Guanosine, Cytidine monophosphate (CMP), malondialdehyde (MDA), Thymidine, Orotic Acid, Nitrate (—NO₃⁻), Uridine monophosphate (UMP), Nicotinamide adenine dinucleotide, oxidized (NAD⁺), Adenosine (ADO), Inosine monophosphate (IMP), Guanosine monophosphate (GMP), Uridine diphosphate-glucose (UDP-Glc), UDP-galactose (UDP-Gal), oxidized glutathione (GSSG), UDP-N-acetyl-glucosamine (UDP-GlcNac), UDP-N-acetyl-galactosamine (UDP-GalNac), Adenosine monophosphate (AMP), Guanosine diphosphate-glucose (GDP-glucose), Cytidine diphosphate (CDP), UDP, GDP, Nicotinamide adenine dinucleotide phosphate, oxidized (NADP⁺), Adenosine diphosphate-Ribose (ADP-Ribose), Cytidine triphosphate (CTP), ADP, Uridine triphosphate (UTP), Guanosine triphosphate (GTP), Nicotinamide adenine dinucleotide, reduced (NADH), Adenosine triphosphate (ATP), Nicotinamide adenine dinucleotide phosphate, reduced (NADPH), Malonyl-CoA, Coenzyme A (CoA-SH), Acetyl-CoA, N-acetylaspartate (NAA).

HPLC Analyses of Free Amino Acids and Amino Group Containing Compounds

The simultaneous determination of primary free amino acids (FAA) and amino group containing compounds (AGCC) (listed below) was performed using the precolumn derivatization of the sample with a mixture of Ortho-phthalaldehyde (OPA) and 3-Mercaptopropionic acid (MPA), as described in detail elsewhere (Amorini et al., Severity of experimental traumatic brain injury modulates changes in concentrations of cerebral free amino acids. J Cell Mol Med. 2017; 21: 530-542; Amorini et al., Metabolic profile of amniotic fluid as a biochemical tool to screen for inborn errors of metabolism and fetal anomalies. Mol Cell Biochem. 2012; 359: 205-216). Briefly, the derivatization mixture composed by 25 mmol/1 OPA, 1% MPA, 237.5 mmol/1 sodium borate, pH 9.8 was prepared daily and placed in the autosampler. The automated precolumn derivatization of the samples (15 μl) with OPA-MPA was carried out at 24° C. and 25 μl of the derivatized mixture were loaded onto the HPLC column (Hypersil C-18, 250×4.6 mm, 5 μm particle size, thermostated at 21° C.) for the subsequent chromatographic separation. In the case of glutamate, deproteinized brain extracts were diluted 20 times with HPLC-grade H₂O prior to the derivatization procedure and subsequent injection. Separation of OPA-AA and OPA-AGCC was carried out at a flow rate of 1.2 ml/min using two mobile phases (mobile phase A=24 mmol/1 CH₃COONa+24 mmol/1 Na₂HPO₄+1% tetrahydrofurane+0.1% trifluoroacetic acid, pH 6.5; mobile phase B=40% CH₃OH+30% CH₃CN+30% H₂O), using an appropriate step gradient (Amorini et al., Severity of experimental traumatic brain injury modulates changes in concentrations of cerebral free amino acids. J Cell Mol Med. 2017; 21: 530-542; Amorini et al., Metabolic profile of amniotic fluid as a biochemical tool to screen for inborn errors of metabolism and fetal anomalies. Mol Cell Biochem. 2012; 359: 205-216).

Assignment and calculation of the OPA-AA and OPA-AGCC in chromatographic runs of whole brain extracts were carried out at 338 nm wavelengths by comparing retention times and areas of peaks with those of peaks of chromatographic runs of freshly-prepared ultra-pure standard mixtures with known concentrations.

List of FAA and ACGC compounds: aspartate (ASP), glutamate (GLU), asparagine (ASN), serine (SER), glutamine (GLN), histidine (HIS), glycine (GLY), threonine (THR), citrulline (CITR), arginine (ARG), alanine (ALA), taurine (TAU), gamma-aminobutyrric acid (GABA), tyrosine (TYR), S-adenosylhomocysteine (SAH), L-cystathionine (L-Cystat), valine (VAL), methionine (MET), tryptophane (TRP), phenylalanine (PHE), isoleucine (ILE), leucine (LEU), ornithine (ORN), lysine (LYS).

Statistical Analysis

Normal data distribution was tested using the Kolmogorov-Smirnov test. Differences across groups were estimated by the two-way ANOVA for repeated measures. Fisher's protected least square was used as the post hoc test. Only two-tailed p-values of less than 0.05 were considered statistically significant

Results

The most evident result among the cerebral values of the 24 standard and non-standard amino acids and primary amino-group containing compounds was that dextran sulfate treatment had a remarkable inhibition of the increase in glutamate (GLU) induced by sTBI (Table 1), thus certainly causing a decrease of excitotocity consequent to excess of this compound.

This effect was, however, visible only if the drug was administered early post-injury (30 min following sTBI), with no efficacy on this excitotoxicity marker when dextran sulfate was injected at 3 days after sTBI. It is also worth underlining that dextran sulfate had significant beneficial effects on compounds involved in the so-called methyl cycle (Met, L-Cystat, SAH), see Table 1.

TABLE 3
concentrations of cerebral compounds

| | ASP | GLU | ASN | SER | GLN | HIS |
|---|---|---|---|---|---|---|
| Control | 2.67 ± 0.45 | 8.95 ± 1.76 | 0.11 ± 0.02 | 0.56 ± 0.14 | 3.70 ± 0.72 | 0.045 ± 0.01 |
| TB 2 | 3.86 ± 0.80 | 11.8 ± 1.15 | 0.12 ± 0.02 | 0.85 ± 0.17 | 4.81 ± 0.78 | 0.060 ± 0.01 |
| days | | | | | | |
| TBI 5 | 3.85 ± 0.91 | 12.77 ± 1.17 | 0.09 ± 0.03 | 0.69 ± 0.19 | 3.57 ± 0.62 | 0.046 ± 0.008 |
| days | | | | | | |
| Acute | 2.40 ± 0.56^{d,i} | 9.81 ± 1.66ⁱ | 0.12 ± 0.02ⁱ | 0.88 ± 0.25^{a} | 4.78 ± 1.09^{a} | 0.068 ± 0.015^{b} |
| phase 1 | | | | | | |
| Acute | 2.94 ± 0.98^{f,j} | 9.93 ± 1.56^{e,i} | 0.13 ± 0.03ⁱ | 0.71 ± 0.28^{b} | 3.66 ± 0.41 | 0.055 ± 0.019 |
| phase 2 | | | | | | |
| Chronic | 4.46 ± 0.70^{a,f} | 13.58 ± 1.28^{a} | 0.18 ± 0.02^{a} | 0.93 ± 0.27^{a,e} | 3.98 ± 0.34 | 0.047 ± 0.021 |
| phase | | | | | | |

| | GLY | THR | CITR | ARG | ALA | TAU |
|---|---|---|---|---|---|---|
| Control | 0.65 ± 0.10 | 0.58 ± 0.15 | 0.018 ± 0.002 | 0.16 ± 0.034 | 0.30 ± 0.067 | 3.60 ± 0.89 |
| TBI 2 | 1.54 ± 0.16 | 0.78 ± 0.17 | 0.017 ± 0.006 | 0.098 ± 0.029 | 0.66 ± 0.17 | 4.93 ± 0.79 |
| days | | | | | | |
| TBI 5 | 0.84 ± 0.13 | 0.60 ± 0.12 | 0.017 ± 0.007 | 0.13 ± 0.52 | 0.35 ± 0.047 | 4.00 ± 0.97 |
| days | | | | | | |
| Acute | 0.83 ± 0.25^{a,c} | 0.92 ± 0.29^{a} | 0.018 ± 0.004 | 0.13 ± 0.02^{b,d} | 0.50 ± 0.12^{a} | 4.86 ± 0.85^{b} |
| phase 1 | | | | | | |
| Acute | 0.71 ± 0.16^{f,i} | 0.66 ± 0.23 | 0.018 ± 0.008 | 0.16 ± 0.03 | 0.52 ± 0.24^{a,e} | 3.80 ± 1.19 |
| phase 2 | | | | | | |
| Chronic | 1.05 ± 0.13^{a,f} | 0.75 ± 0.24^{a,e} | 0.020 ± 0.006 | 0.14 ± 0.02 | 0.57 ± 0.28^{a,e} | 4.49 ± 0.43^{a} |
| phase | | | | | | |

| | GABA | TYR | SAH | L-Cystat | VAL | MET |
|---|---|---|---|---|---|---|
| Control | 1.15 ± 0.40 | 0.120 ± 0.022 | 0.26 ± 0.010 | 0.147 ± 0.080 | 0.049 ± 0.005 | 0.015 ± 0.002 |
| TBI 2 | 1.74 ± 0.35 | 0.160 ± 0.023 | 0.077 ± 0.009 | 0.337 ± 0.011 | 0.057 ± 0.005 | 0.011 ± 0.001 |
| days | | | | | | |
| TBI 5 | 1.50 ± 0.30 | 0.123 ± 0.013 | 0.043 ± 0.013 | 0.202 ± 0.061 | 0.042 ± 0.014 | 0.010 ± 0.001 |
| days | | | | | | |
| Acute | 1.43 ± 0.25^{a} | 0.15 ± 0.03 | 0.033 ± 0.008^{b,c,j} | 0.185 ± 0.031^{b,c,i} | 0.042 ± 0.011 | 0.016 ± 0.005^{d,j} |
| phase 1 | | | | | | |
| Acute | 1.60 ± 0.24^{a} | 0.172 ± 0.046^{b,f} | 0.026 ± 0.010^{f,i} | 0.173 ± 0.038^{b,f,i} | 0.057 ± 0.017 | 0.022 ± 0.006^{b,e,i} |
| phase 2 | | | | | | |
| Chronic | 1.85 ± 0.65^{a} | 0.21 ± 0.05^{f} | 0.050 ± 0.013^{a} | 0.26 ± 0.05^{a,f} | 0.040 ± 0.016^{b} | 0.009 ± 0.004^{b} |
| phase | | | | | | |

| | TRP | PHE | ILE | LEU | ORN | LYS |
|---|---|---|---|---|---|---|
| Control | 0.013 ± 0.002 | 0.023 ± 0.001 | 0.030 ± 0.010 | 0.015 ± 0.002 | 0.012 ± 0.003 | 0.206 ± 0.042 |
| TBI 2 | 0.023 ± 0.004 | 0.046 ± 0.011 | 0.043 ± 0.005 | 0.014 ± 0.007 | 0.013 ± 0.015 | 0.202 ± 0.023 |
| days | | | | | | |
| TBI 5 | 0.012 ± 0.003 | 0.033 ± 0.006 | 0.038 ± 0.010 | 0.014 ± 0.005 | 0.009 ± 0.002 | 0.19 ± 0.092 |
| days | | | | | | |
| Acute | 0.030 ± 0.007^{b,dg,i} | 0.031 ± 0.011^{b,d} | 0.038 ± 0.007 | 0.021 ± 0.005^{a,c} | 0.014 ± 0.007 | 0.236 ± 0.057^{b,d,h} |
| phase 1 | | | | | | |
| Acute | 0.015 ± 0.006 | 0.028 ± 0.010 | 0.048 ± 0.017^{a} | 0.018 ± 0.004 | 0.011 ± 0.005 | 0.32 ± 0.04^{a,e,i} |
| phase 2 | | | | | | |
| Chronic | 0.012 ± 0.007 | 0.033 ± 0.011^{b} | 0.041 ± 0.016^{b} | 0.024 ± 0.032^{b,f} | 0.017 ± 0.009^{a,e} | 0.179 ± 0.036 |
| phase | | | | | | |

^{a}p < 0.01 (comparison with control),
^{b}p < 0.05 (comparison with control),
^{c}p < 0.01 (comparison with TBI 2 days),
^{d}p < 0.05 (comparison with TBI 2 days),
^{e}p < 0.01 (comparison with TBI 5 days),
^{f}p < 0.05 (comparison with TBI 5 days),
^{g}p < 0.01 (comparison with Acute phase 2),
^{h}p < 0.05 (comparison with Acute phase 2),
ⁱp < 0.01 (comparison with Chronic phase),
^{j}p < 0.05 (comparison with Chronic phase)
Table 3 lists the compounds in μmol/g (w.w.)

As is seen in Table 2, dextran sulfate positively affected various compounds related to energy metabolism and mitochondrial functions. Particularly interesting are the concentrations of adenine nucleotides and ATP/ADP ratio as measurement of mitochondrial phosphorylating capacity.

TABLE 2
concentrations of energy metabolites

| | cytosine | creatinine | uracil | β-pseudouridine | cytidine |
|---|---|---|---|---|---|
| Control | 12.89 ± 1.77 | 18.77 ± 2.09 | 10.65 ± 1.11 | 6.32 ± 1.11 | 12.54 ± 1.84 |
| TBI 2 days | 23.58 ± 5.62 | 28.61 ± 3.33 | 17.32 ± 1.54 | 8.45 ± 0.98 | 11.33 ± 1.23 |
| TBI 5 days | 21.56 ± 2.88 | 76.03 ± 8.19 | 24.31 ± 2.60 | 18.66 ± 1.29 | 26.12 ± 2.37 |
| Acute phase 1 | 17.69 ± 2.50^{b,d} | 24.55 ± 3.20^{b,g,i} | 14.56 ± 5.44 | 6.65 ± 1.30^{g,i} | 15.40 ± 3.04 |
| Acute phase 2 | 15.70 ± 4.10^{f} | 37.27 ± 5.82^{a,e,j} | 19.40 ± 7.52^{a,e} | 13.26 ± 3.16^{a,e,j} | 16.18 ± 4.21^{e} |
| Chronic phase | 15.58 ± 2.50^{b,f} | 51.25 ± 10.17^{a,f} | 16.57 ± 2.99^{a,f} | 18.62 ± 2.80^{a} | 14.71 ± 2.83^{e} |

| | hypoxanthine | guanine | xanthine | CDP choline | ascorbic acid |
|---|---|---|---|---|---|
| Control | 7.21 ± 1.22 | 3.12 ± 0.78 | 8.09 ± 1.48 | 7.50 ± 1.01 | 4954.36 ± 212.43 |
| TBI 2 days | 11.36 ± 1.52 | 5.42 ± 0.87 | 13.15 ± 2.88 | 9.83 ± 1.71 | 3186.09 ± 287.87 |
| TBI 5 days | 16.83 ± 2.13 | 4.56 ± 1.29 | 14.14 ± 2.11 | 8.12 ± 1.55 | 2234.51 ± 198.62 |
| Acute phase 1 | 14.47 ± 2.87^{a} | 4.80 ± 1.24^{b} | 9.46 ± 2.34^{d} | 10.93 ± 3.22^{b,h} | 3733.10 ± 277.88^{a,d} |
| Acute phase 2 | 12.90 ± 2.58^{a,j} | 4.73 ± 1.07 | 10.41 ± 2.11^{f} | 6.91 ± 1.86 | 3512.58 ± 224.62^{a,e} |
| Chronic phase | 17.97 ± 4.49^{a} | 5.31 ± 1.04^{b} | 9.35 ± 0.83^{f} | 8.37 ± 2.19 | 3375.03 ± 856.41^{a,e} |

| | uridine | adenine | NO₂ | GSH | inosine |
|---|---|---|---|---|---|
| Control | 56.17 ± 3.88 | 23.14 ± 2.16 | 151.21 ± 16.79 | 3810.29 ± 200.65 | 94.33 ± 17.48 |
| TBI 2 days | 112.09 ± 15.65 | 54.85 ± 8.88 | 233.14 ± 25.48 | 2109.89 ± 156.71 | 126.36 ± 14.06 |
| TBI 5 days | 94.8 ± 10.75 | 76.55 ± 6.33 | 256.28 ± 28.07 | 1902.56 ± 183.42 | 137.73 ± 24.82 |
| Acute phase 1 | 76.35 ± 12.85^{a,c} | 44.82 ± 6.31^{a,d,g} | 216.03 ± 41.74^{a} | 2649.50 ± 397.31^{a,d} | 92.55 ± 31.20^{c} |
| Acute phase 2 | 63.02 ± 9.66^{b,e} | 58.16 ± 6.36^{a,f} | 226.40 ± 30.95^{b} | 2821.50 ± 242.82^{a,e} | 85.52 ± 20.36^{e} |
| Chronic phase | 63.28 ± 3.37^{f} | 52.94 ± 8.59^{a,f} | 217.67 ± 55.04^{a} | 2608.67 ± 358.07^{a,e} | 105.81 ± 25.57^{f} |

| | uric acid | guanosine | CMP | MDA | thymidine |
|---|---|---|---|---|---|
| Control | 2.75 ± 0.35 | 18.96 ± 2.90 | 12.16 ± 1.61 | 1.13 ± 0.25 | 0.54 ± 0.16 |
| TBI 2 days | 30.84 ± 5.13 | 17.52 ± 2.44 | 30.83 ± 4.81 | 28.37 ± 3.37 | 0.67 ± 0.19 |
| TBI 5 days | 23.63 ± 3.40 | 21.32 ± 3.04 | 27.20 ± 3.76 | 7.69 ± 2.18 | 0.97 ± 0.32 |
| Acute phase 1 | 23.62 ± 3.77^{a,d,h} | 20.71 ± 5.66 | 30.12 ± 9.97^{a,h} | 12.47 ± 2.09^{a,c,g} | 0.69 ± 0.11 |
| Acute phase 2 | 19.17 ± 2.15^{a,h,i} | 17.90 ± 3.24^{j} | 15.68 ± 2.12^{f,j} | 4.82 ± 1.73^{a,e,i} | 0.49 ± 0.20^{f} |
| Chronic phase | 27.77 ± 3.60^{a} | 28.87 ± 7.60^{a,f} | 20.51 ± 3.73^{a,f} | 11.62 ± 3.90^{a,e} | 0.71 ± 0.11 |

| | orotic acid | NO₃ | UMP | NAD⁺ | ADO |
|---|---|---|---|---|---|
| Control | 5.67 ± 0.85 | 178.66 ± 37.75 | 96.21 ± 10.51 | 506.88 ± 59.15 | 50.73 ± 8.29 |
| TBI 2 days | 10.09 ± 1.54 | 265.31 ± 47.68 | 116.06 ± 13.55 | 322.37 ± 30.87 | 66.19 ± 11.06 |
| TBI 5 days | 14.27 ± 1.67 | 325.19 ± 60.08 | 128.70 ± 28.28 | 261.67 ± 49.97 | 78.91 ± 20.42 |
| Acute phase 1 | 8.80 ± 2.45^{b,h,j} | 210.64 ± 91.95^{d} | 107.80 ± 21.62 | 404.63 ± 51.10^{a,c,i} | 71.67 ± 15.87 |
| Acute phase 2 | 13.34 ± 3.65^{a} | 198.56 ± 25.93^{e,i} | 138.73 ± 32.01^{b} | 401.18 ± 34.53^{a,e,i} | 82.11 ± 16.51^{a} |
| Chronic phase | 12.05 ± 1.50^{a} | 241.27 ± 18.84^{e} | 103.11 ± 29.79 | 301.13 ± 29.90^{a} | 89.97 ± 12.98^{a} |

| | IMP | GMP | UDP-Glc | UDP-Gal | GSSG |
|---|---|---|---|---|---|
| Control | 54.09 ± 12.15 | 98.93 ± 10.42 | 47.23 ± 3.14 | 120.18 ± 10.99 | 189.21 ± 20.19 |
| TBI 2 days | 50.82 ± 10.45 | 181.94 ± 27.20 | 45.17 ± 6.67 | 131.19 ± 18.49 | 179.51 ± 29.17 |
| TBI 5 days | 124.46 ± 18.97 | 158.35 ± 40.43 | 41.43 ± 5.14 | 112.26 ± 17.36 | 196.65 ± 33.48 |
| Acute phase 1 | 67.71 ± 10.63^{g,i} | 177.00 ± 32.39^{a,g} | 32.14 ± 4.59^{g} | 119.45 ± 12.50 | 185.21 ± 48.10 |
| Acute phase 2 | 102.63 ± 22.09^{a} | 91.47 ± 12.35^{e,i} | 44.44 ± 7.59^{j} | 145.14 ± 27.76 | 219.54 ± 53.36 |
| Chronic phase | 99.29 ± 13.82^{a} | 148.56 ± 31.21^{a} | 35.79 ± 3.45^{b} | 122.29 ± 12.15 | 231.08 ± 44.34^{b,f} |

| | UDP-GlcNac | UDP-GalNac | AMP | GDP glucose | CDP |
|---|---|---|---|---|---|
| Control | 93.71 ± 14.16 | 35.09 ± 3.07 | 30.31 ± 5.12 | 34.89 ± 8.18 | 14.08 ± 1.14 |
| TBI 2 days | 93.71 ± 14.16 | 20.17 ± 3.33 | 73.32 ± 12.88 | 39.16 ± 6.87 | 18.31 ± 2.15 |
| TBI 5 days | 129.54 ± 21.21 | 10.56 ± 2.89 | 98.32 ± 10.99 | 59.88 ± 12.54 | 19.03 ± 6.45 |
| Acute phase 1 | 95.85 ± 19.73^{h,i} | 19.17 ± 4.01^{a} | 53.61 ± 17.91^{a,c,j} | 38.71 ± 6.86 | 25.53 ± 6.83^{a,c} |
| Acute phase 2 | 130.65 ± 28.41^{a} | 19.90 ± 3.12^{a,e} | 57.70 ± 23.01^{a,e,j} | 49.25 ± 10.33^{a} | 24.29 ± 6.76^{a} |
| Chronic phase | 129.42 ± 15.88^{b} | 21.84 ± 2.80^{a,e} | 90.01 ± 21.24^{a} | 43.85 ± 5.06^{b} | 23.55 ± 6.45^{a} |

| | UDP | GDP | NADP⁺ | ADP-ribose | CTP |
|---|---|---|---|---|---|
| Control | 26.06 ± 7.32 | 61.78 ± 17.09 | 27.52 ± 2.58 | 48.88 ± 5.61 | 38.90 ± 4.64 |
| TBI 2 days | 55.47 ± 6.70 | 149.02 ± 19.09 | 16.36 ± 4.41 | 133.31 ± 30.02 | 21.57 ± 3.19 |
| TBI 5 days | 43.71 ± 8.81 | 113.11 ± 28.34 | 12.50 ± 2.97 | 221.80 ± 36.72 | 18.79 ± 3.69 |
| Acute phase 1 | 61.83 ± 10.23^{a,g} | 158.72 ± 24.57^{a} | 17.95 ± 3.28^{a} | 137.87 ± 43.18^{a} | 18.98 ± 6.58^{a,g} |
| Acute phase 2 | 40.38 ± 8.50^{a,i} | 126.70 ± 31.35^{a,j} | 21.27 ± 4.19^{b,e,j} | 141.96 ± 23.56^{a,e,j} | 32.63 ± 3.99^{e,i} |
| Chronic phase | 57.40 ± 5.88^{a,f} | 173.05 ± 28.68^{a,e} | 16.44 ± 2.66^{a,f} | 173.94 ± 8.45^{a} | 25.23 ± 2.93^{a,f} |

| | ADP | UTP | GTP | NADH | ATP |
|---|---|---|---|---|---|
| Control | 233.19 ± 21.33 | 138.95 ± 28.89 | 567.33 ± 54.79 | 14.50 ± 2.75 | 2441.66 ± 257.71 |
| TBI 2 days | 264.71 ± 26.31 | 107.77 ± 12.83 | 208.13 ± 28.36 | 8.54 ± 1.73 | 1350.25 ± 140.87 |
| TBI 5 days | 328.26 ± 31.30 | 90.50 ± 18.69 | 191.81 ± 37.56 | 6.77 ± 1.58 | 1195.81 ± 137.82 |
| Acute phase 1 | 279.34 ± 29.59^{b} | 123.46 ± 15.42^{d} | 255.29 ± 45.21^{a,g} | 15.49 ± 2.05^{c,j} | 1464.25 ± 99.09^{a,h} |
| Acute phase 2 | 264.07 ± 28.29^{b,e,j} | 146.71 ± 32.68^{e} | 336.65 ± 35.18^{a,e,j} | 13.12 ± 4.19^{e} | 1632.23 ± 90.07^{a,e,j} |
| Chronic phase | 315.53 ± 46.53^{a} | 136.80 ± 33.25^{f} | 290.92 ± 34.68^{a,f} | 11.78 ± 3.32^{e} | 1381.03 ± 212.64^{a} |

| | NADPH | malonyl-CoA | CoA-SH | acetyl-CoA | NAA |
|---|---|---|---|---|---|
| Control | 7.95 ± 1.38 | 15.83 ± 1.31 | 28.91 ± 3.19 | 38.97 ± 5.79 | 9141.22 ± 366.64 |
| TBI 2 days | 8.14 ± 1.69 | 10.46 ± 2.56 | 19.64 ± 2.37 | 21.76 ± 4.49 | 5570.00 ± 912.08 |
| TBI 5 days | 9.24 ± 2.07 | 11.89 ± 1.96 | 21.77 ± 1.44 | 18.94 ± 3.75 | 4300.00 ± 480.84 |
| Acute phase 1 | 6.22 ± 1.73 | 12.33 ± 1.82^{b} | 21.61 ± 3.42^{a,h} | 21.56 ± 6.22^{a,g,i} | 6147.91 ± 989.12^{a} |
| Acute phase 2 | 7.05 ± 2.21 | 11.29 ± 2.27^{b} | 30.57 ± 6.02^{f} | 36.86 ± 4.11^{e} | 7262.84 ± 749.73^{a,e} |
| Chronic phase | 7.34 ± 2.65^{f} | 10.00 ± 1.95^{b} | 27.58 ± 6.24^{f} | 35.68 ± 6.55^{e} | 6375.36 ± 974.12^{a,e} |

^{a}p < 0.01 (comparison with control),
^{b}p < 0.05 (comparison with control),
^{c}p < 0.01 (comparison with TBI 2 days),
^{d}p < 0.05 (comparison with TBI 2 days),
^{e}p < 0.01 (comparison with TBI 5 days),
^{f}p < 0.05 (comparison with TBI 5 days),
^{g}p < 0.01 (comparison with Acute phase 2),
^{h}p < 0.05 (comparison with Acute phase 2),
ⁱp < 0.01 (comparison with Chronic phase),
^{j}p < 0.05 (comparison with Chronic phase)
Table 4 lists the compounds in nmol/g (w.w.)

Remarkable changes of oxidative and reduced nicotinic coenzymes were also observed (Table 2).

Parameters related to oxidative stress were also measured and a significant reduction of oxidative stress was detected after administration of dextran sulfate. In particular, ascorbic acid, as the main water-soluble brain antioxidant, and GSH, as the major intracellular-SH donor, were measured. Results showed a significant improvement in their levels after administration of dextran sulfate as shown in Table 2.

In addition, MDA, as end product of polyunsaturated fatty acids of membrane phospholipids and therefore taken as a marker of ROS-mediated lipid peroxidation, was also measured. MDA levels showed a significant reduction after administration of dextran sulfate. The oxidative stress markers described above all indicated an improvement in the recovery of antioxidant status after treatment with dextran sulfate (Table 2).

Indices of representative of NO-mediated nitrosative stress (nitrite and nitrate) were also analyzed. Dextran sulfate administration significantly decreased the nitrate concentrations in both the acute and chronic phases of sTBI (Table 2).

NAA is a brain specific metabolite and a valuable biochemical marker for monitoring deterioration or recovery after TBI. NM is synthesized in neurons from aspartate and acetyl-CoA by aspartate N-acetyltransferase. To ensure NM turnover, the molecule must move between cellular compartments to reach oligodendrocytes where it is degraded into acetate and aspartate by aspartoacylase (ASPA). An upregulation of the catabolic enzyme ASPA and an NAA decrease in order to supply the availability of the substrates asparate and acetyl-CoA are an indication of the status of metabolic impairment. In this study NAA and its substrates were measured after sTBI and showed significant improvements in levels after dextran sulfate administration (Table 2).

These effects on energy metabolites were particularly evident when animals received the dextran sulfate administration early post-injury (30 mins). It is important to note that the overall beneficial effects of dextran sulfate were observed either when the animals were sacrificed 2 days after sTBI or when sacrifice occurred 7 days post sTBI. In these groups of animals, the general amelioration of metabolism connected to AGCC and energy metabolites was more evident, suggesting a long-lasting positive effect of the dextran sulfate administration on brain metabolism.

Discussion

The data presented herein suggests that administration of dextran sulfate reduced levels of glutamate excitotoxicity and ameliorated adverse changes in metabolic homeostasis by protecting mitochondrial function, indicating a neuroprotective effect of the compound after severe TBI.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

1. Dextran sulfate, or a pharmaceutically acceptable salt thereof, for use in treatment or prevention of muscle atrophy in a subject suffering from sarcopenia.

2. Dextran sulfate, or a pharmaceutically acceptable salt thereof, for use in improving muscle function in a subject suffering from a neuromuscular disease and/or damage or sarcopenia.

3. Dextran sulfate, or the pharmaceutically acceptable salt thereof, for use according to claim 2, wherein the subject is suffering from an intrinsic muscle disease and/or damage.

4. Dextran sulfate, or the pharmaceutically acceptable salt thereof, for use according to claim 3, wherein the intrinsic muscle disease and/or damage is selected from the group consisting of muscular dystrophy, Duchenne muscular dystrophy (DMD), amyotrophic lateral sclerosis (ALS) and myositis.

5. Dextran sulfate, or the pharmaceutically acceptable salt thereof, for use according to claim 2, wherein the subject is suffering from a central and/or peripheral nervous system disease and/or damage.

6. Dextran sulfate, or the pharmaceutically acceptable salt thereof, for use according to claim 5, wherein the central and/or peripheral nervous system disease and/or damage is selected from the group consisting of stroke, spinal cord injury, traumatic brain injury (TBI), cerebral palsy, Charcot-Marie-Tooth disease (CMT), primary lateral sclerosis (PLS), spinal muscular atrophy (SMA), nerve entrapment, and a surgical complication.

7. Dextran sulfate, or the pharmaceutically acceptable salt thereof, for use according to any of the claims 1 to 6, wherein the dextran sulfate, or the pharmaceutically acceptable salt thereof, is formulated for systemic administration to the subject.

8. Dextran sulfate, or the pharmaceutically acceptable salt thereof, for use according to claim 7, wherein the dextran sulfate, or the pharmaceutically acceptable salt thereof, is formulated for intravenous or subcutaneous administration to the subject, preferably formulated for subcutaneous administration to the subject.

9. Dextran sulfate, or the pharmaceutically acceptable salt thereof, for use according to any of the claims 1 to 8, wherein the dextran sulfate, or the pharmaceutically acceptable salt thereof, has an average molecular weight equal to or below 10 000 Da.

10. Dextran sulfate, or the pharmaceutically acceptable salt thereof, for use according to claim to 9, wherein the average molecular weight is within a range of from 2 000 to 10 000 Da, preferably within a range of from 3 000 to 10 000 Da, and more preferably within a range of from 3 500 to 9 500 Da.

11. Dextran sulfate, or the pharmaceutically acceptable salt thereof, for use according to claim to 10, wherein the average molecular weight is within a range of from 4 500 to 7 500 Da, preferably within a range of from 4 500 to 5 500 Da.

12. Dextran sulfate, or the pharmaceutically acceptable salt thereof, for use according to any of the claims 1 to 11, wherein the dextran sulfate, or the pharmaceutically acceptable salt thereof, has an average sulfur content in a range of from 15 to 20%.

13. Dextran sulfate, or the pharmaceutically acceptable salt thereof, for use according to claim 12, wherein the dextran sulfate, or the pharmaceutically acceptable salt thereof, has an average sulfur content of about 17%.

14. Dextran sulfate, or the pharmaceutically acceptable salt thereof, for use according to any of the claims 1 to 9, wherein the dextran sulfate, or the pharmaceutically acceptable salt thereof, has a number average molecular weight (Mₙ) as measured by nuclear magnetic resonance (NMR) spectroscopy within a range of from 1850 to 3500 Da, preferably within a range of from 1850 to 2500 Da, and more preferably within a range of from 1850 to 2300 Da.

15. Dextran sulfate, or the pharmaceutically acceptable salt thereof, for use according to claim 14, wherein the dextran sulfate, or the pharmaceutically acceptable salt thereof, has a Mₙ as measured by NMR spectroscopy within a range of from 1850 to 2000 Da.

16. Dextran sulfate, or the pharmaceutically acceptable salt thereof, for use according to claim 14 or 15, wherein the dextran sulfate, or the pharmaceutically acceptable salt thereof, has an average sulfate number per glucose unit within a range of from 2.5 to 3.0, preferably within a range of from 2.5 to 2.8, and more preferably within a range of from 2.6 to 2.7.

17. Dextran sulfate, or the pharmaceutically acceptable salt thereof, for use according to any of the claims 1 to 16, wherein the dextran sulfate, or the pharmaceutically acceptable salt thereof, has on average 5.1 glucose units and an average sulfate number per glucose unit of 2.6 to 2.7.

18. Dextran sulfate, or the pharmaceutically acceptable salt thereof, for use according to any of the claims 1 to 17, wherein the dextran sulfate, or the pharmaceutically acceptable salt thereof, is formulated as an aqueous injection solution.

19. Dextran sulfate, or the pharmaceutically acceptable salt thereof, for use according to any of the claims 1 to 18, wherein the pharmaceutically acceptable salt thereof is a sodium salt of dextran sulfate.
